(19) United States
(12) Patent Application Publication
(10) Pub. No.: US 2003/0224480 A1
(43) Pub. Date: Dec. 4, 2003

(54) Method of designing multifunctional base sequence

(75) Inventors: Yoko Satou, Fukuoka-shi (JP); Masato Kitajima, Fukuoka-shi (JP); Kiyotaka Shiba, Tokyo (JP)

Correspondence Address: STAAS & HALSEY LLP, SUITE 700, 1201 NEW YORK AVENUE, N.W., WASHINGTON, DC, 20005, US

(73) Assignee: FUJITSU LIMITED, Kawasaki (JP)

(21) Appl. No.: 10/329,781

(22) Filed: Dec. 27, 2002

(30) Foreign Application Priority Data
Dec. 27, 2001 (JP) JP2001-397390

(51) Int. Cl. C12P 21/02; C12N 5/06; C07K 7/08; G06F 19/00; G01N 33/48; G01N 33/50; C07H 21/04

(52) U.S. Cl. 435/69.1; 435/320.1; 435/325; 530/324; 536/23.5; 702/19; 530/331

(57) ABSTRACT

To provide a method of designing a multifunctional base sequence which can largely shorten the calculation time and reduce the volume of memory consumption of a processor by carrying out calculation with the advance exclusion of base sequences in which translation termination codons are emerged in the second and third reading frames which are to be excluded in the end. Focusing on the fact that a dipeptide sequence already contains information about the translation products of the second and third reading frames, proteins are analyzed and calculated as duplicated connective products of dipeptide sequences, and not analyzed as connective products of 20 kinds of amino acids. In “Leu-Ser” case, for example, calculation may only be performed hereafter for 6×6−10=26 variants that do not contain termination codons in the second and third reading frames (FIG. 1). Further, in the case of “Leu-Ser-Arg” sequence, by selecting the combinations having the same codon for serine from 26 variants of “Leu-Ser” 6-mer codons and from 32 variants of “Ser-Arg” 6-mer codons, and connecting them, from now on, calculation would be performed only for 142 variants out of 218 variants, and connected.

### TECHNICAL FIELD

The present invention relates to the field of computational science for designing a multifunctional base sequence (a multifunctional microgene) which is associated with biological functions in a plurality of reading frames, and to the field of protein engineering for producing an artificial protein by using the multifunctional base sequence.

### BACKGROUND ART

Knowledge concerning structures and functions of proteins obtained from genomic biology and post genomic biology can now be artificially reorganized on artificial proteins and actively utilized. As a method of rationally embedding a function on an artificial protein, a small base sequence (a microgene) is first designed to associate with a specific biological function, and then it is possible to reorganize the specific biological function on an artificial protein which is a translation product of a microgene polymer by polymerizing the microgene in a tandem manner (Proc. Natl. Acad. Sci. USA 94, 3805-3810, 1997, Japanese Laid-Open Patent Application No.1997-322775), or by connecting plural microgenes (Japanese Laid-Open Patent Application No.1997-154585). There is, for example, a method of microgene polymerization (Proc. Natl. Acad. Sci. USA 94, 3805-3810, 1997, Japanese Laid-Open Patent Application No.1997-322775) to polymerize microgenes, which has an aspect that different translation reading frames of the microgenes are utilized in parallel. It is indispensable for the development of high-function artificial proteins to design and utilize a “multifunctional base sequence” which is embedded with a plurality of biological functions simultaneously in a plurality of reading frames, by taking advantage of this aspect of the microgene polymerization method (Japanese Patent Application No.2000-180997).

To present, designing of such multifunctional base sequence underwent the process as follows: to set a given peptide sequence having a primary function as an initial value; to back-translate base by base to the base sequences according to a genetic code table; to create all base sequences capable of encoding the peptide sequence on the processor; then to write down a pool of peptide sequences which are encoded by all the base sequences created and which are arising from reading frames different from that of the first peptide sequence in the processor; and lastly to select peptides having the secondary and tertiary functions out of this pool of peptide sequences.

In this case, base sequences in which translation termination codons emerge in other reading frames at the junction points of residues in a peptide of the first reading frame also become objects of the calculation. Such base sequences accompanied with emergence of translation termination codons in other reading frames have to be excluded in the end from the standpoint of applicability of multifunctional genes. However, it was hard to exclude the base sequences in advance in a conventional algorithm as described above so that all the combinations had to be calculated, which required vast amount of calculation time. For example, there are approximately 687×10⁸ variants of base sequences encoding the peptide sequence of NGNNGNNGNNGNNGNNGNGNNGNNGG in its first reading frame, and among them only about 4×10⁷ variants are devoid of translation termination codons in the second and third reading frames. In the conventional method, however, all the variants of about 687×10⁸ had to undergo calculation.

The subject of the present invention is to provide a method of designing a multifunctional base sequence wherein the calculation time is largely shortened and the volume of memory consumption of a processor is largely reduced by calculating with the advance exclusion of base sequences which are accompanied with the emergence of translation termination codons in the second and third reading frames, and which should be excluded in the end.

The present inventors have made a keen study to solve the above described subject and focused on the fact that a dipeptide sequence (two amino acid residues) or a peptide sequence with longer length already contains information about translation products in the second and third reading frames. Then the present inventors have found that, when proteins are analyzed and calculated by regarding proteins as the duplicated and connective products of dipeptide sequences (two amino acid residues) or of short sequences with length longer than dipeptides unlike in conventional methods where proteins are analyzed as connective products of 20 kinds of amino acids, the information can be analyzed in such a way as the information of translation products of the second and third reading frames is included within, and therefore the calculation time is largely shortened and the volume of memory consumption of a processor can be reduced to a great extent.

FIG. 1 shows an example of the course of processing to back-translate into base sequences by single amino acid units. For instance, there are six codons encoding leucine (Leu); TTA, TTG, CTT, CTC, CTA and CTG. There are also six codons encoding serine (Ser); TCT, TCC, TCA, TCG, AGT and AGC. To perform back translation for all base sequences that are capable of encoding a dipeptide “Leu-Ser”, 6×6=36 variants of base sequences are first generated on the processor. Besides, for the case of the sequence “Leu-Ser-Arg” where arginine (Arg) is located on the third position, 36×6=216 variants of base sequences are generated on the processor. In this way, variants of base sequences corresponding to the total variants obtained by multiplying codons (1-6 variants) which have possibility for encoding the amino acid located at the Nth position are generated on the processor, and then the processing moves on to the exclusion of base sequences containing translation termination codons (TAA, TAG, TGA) in other reading frames from among the base sequences. Since a base sequence containing a translation termination codon in other reading frames cannot be used as a multifunctional base sequence in the end, the exclusion of them at this stage will largely reduce the burden on the later calculation processing.

Next, a processing is considered under the recognition that a polypeptide sequence is a pool of 400 dipeptide variants and not a connection of 20 amino acid residues. When considering a base sequence which encodes a dipeptide, the first amino acid residue of the second and third reading frames in the base sequence are already defined in the first place. Therefore, it becomes possible to exclude in advance the sequences containing termination codons out of the pool of base sequences encoding a dipeptide. As shown in the aforementioned FIG. 1, there are eight sequences containing termination codons in the second reading frames and two sequences containing termination codons in the third reading frames among all 36 variants of base sequences capable of encoding the dipeptide “Leu-Ser”. Therefore, it becomes possible to generate base sequences on the processor with the advance exclusion of termination codons by preparing 36−10=26 variants as codons corresponding to “Leu-Ser”.

For example, when carrying out back-translation for a peptide comprising three residues of “Leu-Ser-Arg” and generating base sequences encoding the peptide in a processor, the sequence is processed as a sequence where two dipeptides, “Leu-Ser” and “Ser-Arg”, are connected. Codons corresponding to “Leu-Ser” may thereafter be calculated for 6×6−10=26 variants as described above, and codons corresponding to “Ser-Arg” may be calculated for 6×6−4=32 variants (four variants contain termination codons in their second reading frames). Therefore, as shown in FIG. 2, it has become possible to obtain every base sequence with the length of 9-mer which encodes “Leu-Ser-Arg” in the first reading frame and not containing termination codons in the second and third reading frames by selecting and connecting the codon combinations, where serine is read by the same codon, from 26 variants of “Leu-Ser” 6-mer codons and from 32 variants of “Ser-Arg” 6-mer codons. As a result of this, (6×4)+(6×6)+(6×6)+(6×6)+(1×4)+(1×6)=142 variants would just be enough to be processed and calculated as shown in FIG. 2, whereas codon combinations according to the conventional methods required work of writing down sequences of 6×6×6=216 variants on a processor.

As described in the foregoing, an operation in which processing for the sequences which would finally be excluded due to the emergence of termination codons can be avoided by processing a polypeptide sequence as a pool of dipeptide units, preferably as a pool of sequential dipeptide units with duplicated amino acid residues, and by preparing a dipeptide-codon corresponding table (a corresponding table for nucleic acid sequences encoding dipeptides) where those having termination codons in the second and third reading frames are excluded in advance from codons of the dipeptide units. In fact, utilization of such algorithm enables the calculation time to be largely shortened as described later. Furthermore, it enables the necessary memory size to be also reduced to a great extent.

Besides, when a dipeptide-codon table, in which termination codons are excluded in advance, is translated in three reading frames, a sort of the first amino acids in the second and third reading frames are proved to be defined in the first place as FIG. 3 indicates. For example, the first reading frame TTA in the sequence of TTATCT for “Leu-Ser” is leucine (L), however, it is defined in the first place that the first amino acid in the second reading frame is tyrosine (Y) encoded by TAT, and the first amino acid in the third reading frame is isoleucine (I) encoded by ATC. Therefore, having given a dipeptide, thinkable sorts of amino acids in the second and third reading frames at that position are defined in the first place without back-translating to base sequences for each time. A considerable reduction in calculation processing can become possible by preparing in advance a “corresponding table for amino acids for each dipeptide-reading frame” to avoid the processing of back-translation to the base sequences. In this case, however, necessary information for connecting the first and the second dipeptide informations, as found in FIG. 2, is not included, and thus some extra information are needed for acquiring information about the possible “combinations”. Nevertheless, sufficient amount of information can be yielded for finding out the sorts of amino acids that can be emerged in the second and third reading frames and for obtaining knowledge of their rough existing ratios when starting from a given peptide sequence in the first reading frame.

Information concerning the amino acid combinations which can be emerged in the second and third reading frames can also be given by further providing information of the kinds of codon used, for instance, to the aforementioned “corresponding table for amino acids for each dipeptide-reading frame”. This turns out to be the same substance as the back-translation processing to the base sequences demonstrated in FIG. 2, yet it is characterized in that the volume of memory consumption can be reduced and the processing in which other information, such as information of the usage frequency of codons, is embedded can be performed.

The present invention has come to the completion based on the findings described above.

### DISCLOSURE OF THE INVENTION

The present invention relates to: a method of designing a multifunctional base sequence wherein a base sequence has two or more functions in different reading frames of said base sequence, wherein a protein or a peptide encoded by a base sequence arising from one of the three reading frames is processed as a pool of oligopeptide units, and wherein the base sequence information of other reading frames contained in the oligopeptide sequence is utilized (claim 1); the method of designing a multifunctional base sequence according to claim 1, wherein a corresponding table for nucleic acid sequences encoding oligopeptide sequences is produced and used (claim 2); the method of designing a multifunctional base sequence according to claim 1 or 2, wherein a processing is carried out for a pool of sequential oligopeptide units having duplicated amino acid residues, and wherein a processing is carried out to connect oligopeptide units that have same codon for the duplicated amino acid residue in the sequential oligopeptide units (claim 3); the method of designing a multifunctional base sequence according to claim 1 or 2, wherein a processing is carried out to connect amino acid residues encoded by base sequences of other reading frames contained in the oligopeptide units (claim 4); the method of designing a multifunctional base sequence according to any of claims 1-4, wherein the processing for a pool of oligopeptide units is a processing to exclude base sequences containing termination codons from among the base sequences of other reading frames contained in the oligopeptide units (claim 5); the method of designing a multifunctional base sequence according to any of claims 1-4, wherein the processing for a pool of oligopeptide units is a processing to select the whole or a part of a sequence of the interest from among the base sequences of other reading frames contained in the oligopeptide units (claim 6); the method of designing a multifunctional base sequence according to any of claims 1-6, wherein the base sequence is a double-stranded base sequence (claim 7); and the method of designing a multifunctional base sequence according to any of claims 1-7, wherein the oligopeptide units are dipeptide units or tripeptide units (claim 8).

The present invention further relates to: a method of generating a multifunctional base sequence having two or more functions wherein the method of designing a multifunctional base sequence according to any of claims 1-8 is employed (claim 9); and a method of generating an artificial protein wherein the method of designing a multifunctional base sequence according to any of claims 1-8 is employed (claim 10).

### BRIEF DESCRIPTION OF DRAWINGS

FIG. 1 is an example of an algorithm for designing a base sequence encoding a dipeptide (Leu-Ser) which is devoid of termination codons in the second and third reading frames.

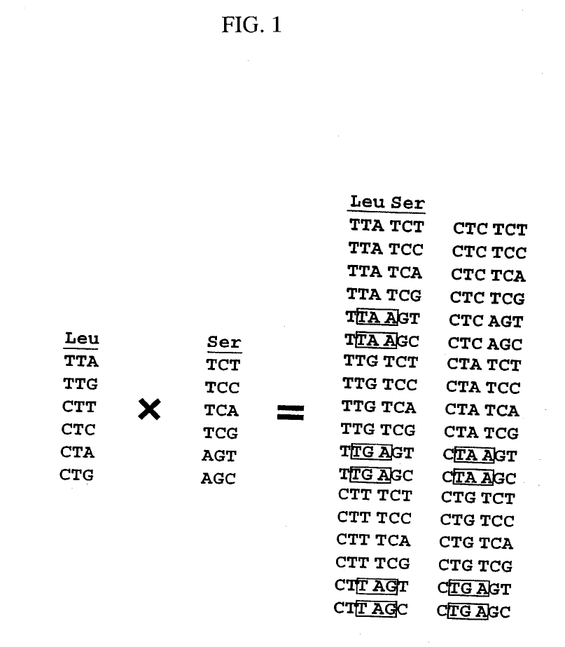

FIG. 2 is an example of an algorithm for designing a base sequence encoding a tripeptide (Leu-Ser-Arg) which is devoid of termination codons in the second and third reading frames.

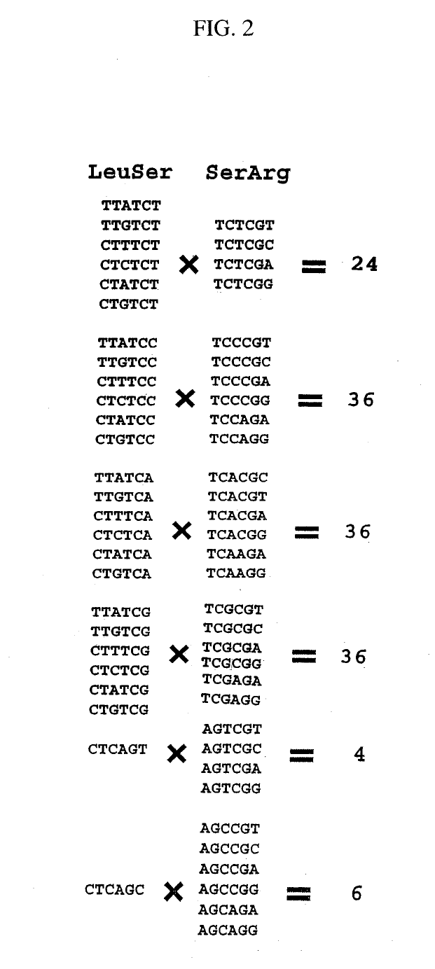

FIG. 3 shows that a sort of the first amino acid in the second and third reading frames are defined in the first place by translating in three reading frames a dipeptide (Leu-Ser) codon table which is devoid of termination codons in the second and third reading frames.

FIG. 4 shows a codon table where the first amino acid of dipeptides is A (alanin) among the dipeptide-codon tables.

FIG. 5 shows a processing flow chart illustrating the method of designing a multifunctional base sequence of the present invention.

### BEST MODE OF CARRYING OUT THE INVENTION

There is no particular limitation as to a method of designing a multifunctional base sequence of the present invention as long as it is a method of designing a multifunctional base sequence: wherein a base sequence has two or more functions in different reading frames of the base sequence; wherein proteins or peptides (usually, these proteins or peptides are given as translation products of the first reading frame), which are encoded by the base sequence deriving from one of three reading frames, are processed as a pool of oligopeptide units, preferably as a pool of dipeptide units; and wherein the base sequence information of other reading frames contained in oligopeptide sequences, preferably in dipeptide sequences, is utilized. However, it is preferable to produce in advance a corresponding table for nucleic acid sequences encoding oligopeptide sequences represented by a corresponding table for nucleic acid sequences encoding dipeptide sequences (a dipeptide-codon corresponding table), and to use the corresponding table. In this description, an oligopeptide means a peptide in which 2-8 amino acid residues are connected.

Combinations of dipeptide codons count 3721 ways which is square of 64-3, among which 192 ways of combination are accompanied with the emergence of termination codons respectively in the second and third reading frames. This means 384/3721=10% plus can be excluded in advance from the calculation objects by constructing a dipeptide-codon corresponding table. For example, 10/36 in “Leu-Ser” and 4/36 in “Ser-Arg” will be excluded in advance from the calculation objects as described earlier. For instance, leucine-threonine “Leu-Thr” is exemplified as a dipeptide sequence containing many combinations to be excluded from the calculation objects. Among 6×4=24 codon combinations for “Leu-Thr”, 16 combinations (TTA ACT; TTA ACC; TTA ACA; TTA ACG; TTG ACT; TTG ACC; TTG ACA; TTG ACG; CTAACT; CTAACC; CTAACA; CTAACG; CTGACT; CTGACC; CTGACA; CTGACG) are subjected to cancellation of calculation due to the termination codons, and calculation will be continued for 8 combinations (CTT ACT; CTT ACC; CTTACA; CTTACG; CTCACT; CTCACC; CTCACA; CTCACG), meaning that as much as 2/3 is excluded from the calculation objects in advance. Besides, in methionine-isoleucine “Met-Ile”, all three kinds (ATGATT; ATGATC; ATGATA) come to possess a termination codon TGA in the second reading frame and are excluded from the calculation objects, therefore, calculation time can largely be shortened by checking in advance whether a given amino acid sequence for a protein or a peptide contains the “Met-Ile” dipeptide sequence.

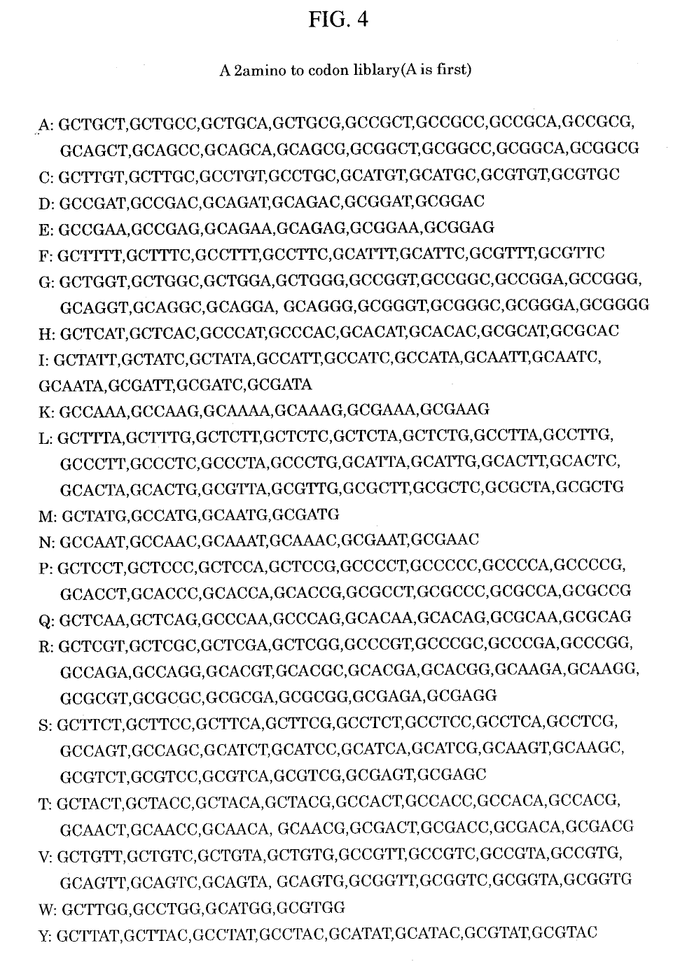

Codon tables indicating the case where calculation is cancelled in the course of a program can be made the above-described dipeptide-codon corresponding table, however, it is usually sufficient to produce and prepare codon tables for 400 kinds indicating the case where calculation continues in the course of a program. Such codon tables may be, for example, produced for each of the first amino acids of dipeptides. Among dipeptide-codon tables, FIG. 4 displays 20 kinds of codon tables where the first amino acid of dipeptides is A (alanine), in the sequential order of AA, AC, AD, . . . , and so on.

In the method of designing a multifunctional base sequence of the present invention, it is preferable to carry out a processing for sequential oligopeptide units with duplicated amino acid residues, preferably for a pool of dipeptide units, and to perform a processing to connect dipeptide units having same codon for the duplicated amino acid residue in the sequential dipeptide units. Construction of an oligopeptide-codon corresponding table is enabled by the use of this algorithm. For example, as described earlier, when a peptide comprising three residues of “Leu-Ser-Arg” is back-translated and base sequences encoding the peptide are generated on the processor, the sequence is regarded as a sequence in which two dipeptides “Leu-Ser” and “Ser-Arg” are connected. Therefore, by connecting and processing the dipeptide units having the same codon for serine which is a duplicated amino acid residue, a codon corresponding table for tripeptide “Leu-Ser-Arg” can be produced and by using this codon corresponding table for the tripeptide “Leu-Ser-Arg”, 74 variants are excluded and the objects for processing and calculation can be reduced to 142/216. Likewise, in the case of “Leu-Thr-Lys”, the sequence is regarded as a connection of two dipeptides, “Leu-Thr” and “Thr-Lys”, and dipeptide units having the same codon for threonine, a duplicated amino acid residue, are connected and processed to reduce the objects to 12/48. Furthermore, in the case of “Leu-Arg-Ser”, the sequence is regarded as a connection of two dipeptides, “Leu-Arg” and “Arg-Ser”, and dipeptide units having the same codon for arginine, a duplicated amino acid residue, are connected and processed to reduce the objects for processing and calculation to 144/216. Hence corresponding tables for oligopeptide units which are longer than tetrapeptide units can be constructed.

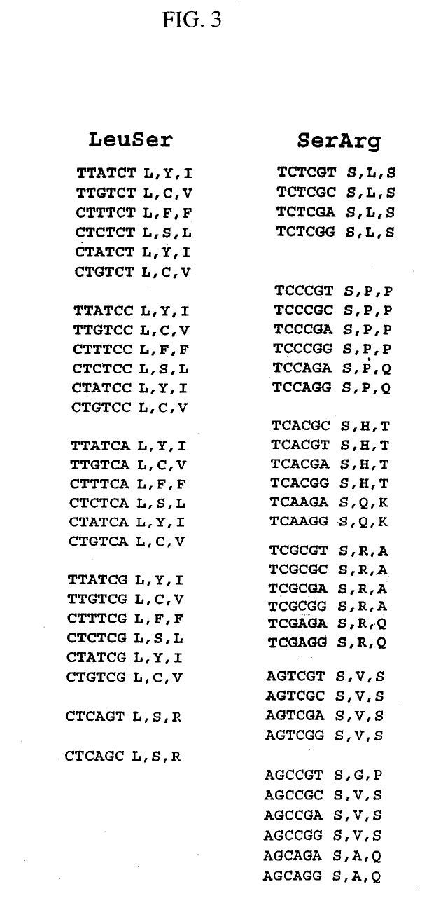

In the method of designing a multifunctional base sequence of the present invention, a processing can be carried out to connect amino acid residues which are encoded by base sequences of other reading frames contained in oligopeptide units, preferably in dipeptide units. Taking the dipeptide combination “Leu-Ser” (a case for LS) shown in FIG. 3 as an example, kinds of amino acids which can emerge in the second reading frame are C, F, S and Y when starting from a given peptide sequence of the first reading frame, whereas those which can emerge in the third reading frame are F, I, L, R and V. By utilizing the algorithm which employs such “corresponding table for amino acid sequence for each dipeptide-reading frame”, approximate existing ratios of amino acid residues capable of emerging in the second or third reading frame can be acquired which are as follows: C;8 (8/26=0.31), F;4 (4/26=0.15), S;6 (6/26=0.23) and Y;8 (8/26=0.31) in the second reading frame, and F;4 (4/26=0.15), I; 8 (8/26=0.31), L;4 (4/26=0.15), R;2 (2/26=0.08) and V;8 (8/26=0.31) in the third reading frame.

Other than the processing to exclude base sequences including termination codons from the base sequences of other reading frames contained in oligopeptide units, preferably in dipeptide or tripeptide units, it is possible to carry out a processing to select base sequences containing the whole or a part of the sequence of the interest in the method of designing a multifunctional base sequence according to the present invention. Although the processing to select sequences of the interest is preferably carried out for the base sequences where termination codons have been excluded, it can also be carried out for the base sequences where termination codons have not been excluded. Such sequence of the interest is exemplified by a sequence with a function of the interest, and such function of the interest may roughly be grouped into: functions possessed by translation products of the whole or a part of the base sequence; and functions of the whole or a part of the base sequence per se.

The functions possessed by translation products as mentioned above include: function to easily form secondary structures such as α-helix-formation or the like; antigen function to induce neutralizing antibodies for such as virus or the like; function to activate immunity (Nature Medicine, 3: 1266-1270, 1997); function to promote or suppress cell proliferation; function to specifically recognize cancer cells; protein transduction function; cell-death-inducing function; function to present residues that determine antigens; metal-binding function; coenzyme-binding function; function to activate catalysts; function to activate fluorescence signal; function to bind to a specific receptor and to activate the receptor; function to bind to a specific factor involved in signal transduction and to modulate the action of the factor; function to specifically recognize biopolymers such as proteins, DNA, RNA, sugar or the like; cell adhesion function; function to localize proteins to the cell exterior; function to target at a specific intracellular organelle (mitochondrion, chloroplast, ER, etc.); function to be embedded in the cell membrane; function to form amyloid fibers; function to form fibrous proteins; function to form a protein gel; function to form a protein film; function to form a single molecular membrane; self-aggregation function; function to form particles; function to assist the formation of higher-order structure of other proteins; function to recognize inorganic crystals; function to suppress the growth of inorganic crystals; and the like. As for the functions of the base sequence per se as described above are exemplified by the followings: metal-binding function; coenzyme-binding function; function to activate catalysts; function to bind to a specific receptor and to activate the receptor; function to bind to a specific factor involved in signal transduction and to modulate the action of the factor; function to specifically recognize biopolymers such as proteins, DNA, RNA, sugar or the like; function to stabilize RNA; function to modulate the translation efficiency; function to suppress the expression of a specific gene; and so on.

There is no specific limitation as to a method of producing a multifunctional base sequence according to the present invention as long as it is a method of producing a multifunctional base sequence which comprises a process of selecting base sequences having two or more functions by using the method of designing a multifunctional base sequence of the present invention, and any base sequence having two or more functions in different reading frames of the base sequence can be an object of such multifunctional base sequence, where a base sequence is specifically exemplified by single- or double-stranded DNA or RNA sequences. These sequences can either take linear or cyclic structure, however, a sequence with linear structure is preferable because a polymerization method for a linear structured sequence has been established. Furthermore, it is preferable that the aforementioned multifunctional base sequence is devoid of termination codons in all three reading frames where the reading frames are shifted one-by-one within the base sequence, and especially for a double-stranded base sequence, it is preferable that all six reading frames in the base sequence are devoid of termination codons. Still further, such base sequence is particularly preferable that a termination codon will not emerge at the junction points (binding points) arising from the polymerization of the multifunctional base sequence.

The length of a multifunctional base sequence of the present invention will not be limited to a particular length. However, base sequences consisting of 15-500 bases or base pairs, particularly, 15-200 bases or base pairs, and more particularly, 15-100 bases or base pairs are preferable for a stable performance of DNA synthesis. Further, the following multifunctional base sequences may be used as a multifunctional base sequence of the present invention: a multifunctional base sequences which is modified for polymerization according to formation of random polymer of microgene (Publication of Japanese Laid-Open Patent Application No.1997-154585) or the method of microgene polymerization (Publication of Japanese Laid-Open Patent Application No.1997-322775) as described earlier, or by some other methods; and a multifunctional base sequence to which a natural base sequence is bound.

Base sequences having biological functions that are same as or different from the given functions can be selected by the computational science approach utilizing a computer. These approaches are exemplified more specifically by an approach in which selection is made using scores obtained by a biological function prediction program. Such biological function prediction program is exemplified by a program produced by statistically processing the correlations between biological functions of proteins and peptides and the primary structure of proteins and peptides. The potential for secondary structure formation of a peptide, for instance, can be assessed by using a previously reported protocol (Structure, Function, and Genetics 27: 36-46, 1997). By using this method, the possibility of α-helix- and β-strand-formation predicted at the each residue position of the given peptide sequences is numerically displayed (larger values for higher possibility). The potential levels for α-helix- and β-strand-formation at all the residues of the given peptide sequences are totaled respectively and calculated as a probability of α-helix-formation of the given peptide and a probability of β-strand-formation of the given peptide, and then can be used for the assessment. Other than the above, the following programs are exemplified as function prediction programs: protein family data basis such as “Motiffind program” (Protein Sci., 5: 1991-1999, 1996) and the like for detecting the similarities to known motifs registered to, for example, “PROSITE” (Nucleic Acids Res., 27: 215-219, 1999); a similarity searching program “blast” for predicting functions based on the similarities to natural proteins (J. Mol. Biol., 215: 403-410, 1990); “SMART” program for calculating the similarities to various protein factors of the signal transduction system (Proc. Natl. Acad. Sci. USA, 95: 5857-5864, 1998); “PSORT” program for assessing the potential to localize proteins to the cell exterior or to intracellular organelles (Biochem. Sci., 24: 34-35, 1999); “SOSUI” program for assessing the potential to be embedded in the cell membrane (Bioinformatics, 4: 378-379, 1998); and so on.

Sequences obtained by binding two or more multifunctional base sequences of the different kinds with ligase or the like, or by binding a multifunctional base sequence to a natural base sequence with ligase or the like can be adopted as a multifunctional base sequence of the present invention. Further, a sequence obtained by separately producing the parts of the multifunctional sequence of the present invention and then binding these parts with ligase or the like can also be adopted as a multifunctional base sequence of the present invention. Still further, a sequence having two or more functions produced by the method of producing a multifunctional base sequence of the present invention as described above is also included in the multifunctional base sequence of the present invention.

There is no particular limitation as to a method of producing an artificial protein of the present invention as long as the method comprises: by using the method of designing a multifunctional base sequence of the present invention and from among all the combinations of base sequences encoding an amino acid sequence having a given function, selecting an artificial gene comprising a base sequence having a function same as or different from the aforementioned given function in the second and third reading frames which are different from that of the amino acid sequence having the aforementioned given function; and generating an artificial protein based on the sequence information of the artificial gene. However, the aforementioned biologic functions are preferable for a given function, and a biological function different from the given function is preferable in that diversity can be yielded. The above-mentioned amino acid sequence having a given function is covered by every amino acid sequence having a given function and will not be limited to a single amino acid sequence. For instance, if there are three amino acid sequences having a given function, a multifunctional base sequence will be selected out of all the combinations of base sequences encoding the three amino acid sequences. Other than the known sequences such as, for example, a sequence of the aforementioned neutralizing antigen for AIDS virus or a motif structure such as Glu-Leu-Arg or the like held by the α-chemokine which is a cytokine to leukemia, the following unknown sequences are exemplified as an amino acid sequence having such given function: a sequence arising from deletion, substitution or addition of one or more amino acids in the known sequences and having similar functions to those of the known sequences; a common sequence well preserved among organisms, which is involved in a specific biological function; and a sequence comprising an amino acid sequence avoided by an existing human protein, which has possibility of evading the surveillance of the human immune system.

The present invention will be explained in more detail below with reference to the examples. However, the scope of the invention will not be limited to these examples.

### EXAMPLE 1

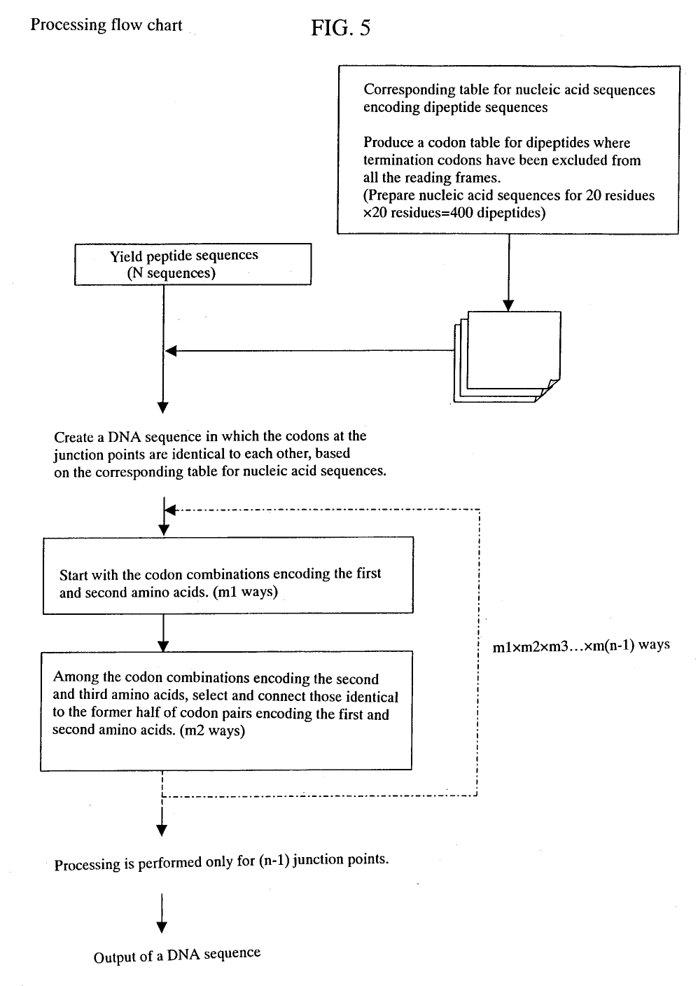

A primary sequence NGNNGNNGNNGNNGNNGNGNNGNNGG (S1) was given and among base sequences which encode this peptide sequence consisting of asparagine (N) and glycine (G), those not containing termination codons were generated on the processor according to the processing flow chart shown in FIG. 5. The number of total patterns of base sequences encoded in the first reading frame of this peptide sequence counts as much as 687×10⁸ variants approx., and in conventional methods all of such base sequences were processed. However, by adopting the algorithm using the “nucleic acid sequence-dipeptide corresponding table” of the present invention, processing is only required for 4×10⁷ variants approx. which do not contain translation termination codons in the second and third reading frames. As a result of this, the calculation time was shortened to about 15 min when the algorithm of the present invention was applied, in contrast to the fact that it took about two weeks for the calculation time in conventional methods. Owing to this, vain calculation processing which equals to about 99.95% of the total patterns can be avoided. A computer employing the specification of OS: Solaris2.7, CPU: Ultra SPARC-II was used for the calculation.

### EXAMPLE 2

Similarly as in Example 1, a primary sequence YNGDNGNNGDNGNNG (S2) was given and DNA sequences encoding this peptide sequence were generated on the processor. The total patterns of base sequence variants encoded in the first reading frame were approximately 1×10⁶. However, when the algorithm according to the “nucleic acid sequence-dipeptide corresponding table” of the present invention was applied, it was proved that the processing should only be carried out for about 1×10⁴ variants that had no translation termination codons in the second and third reading frames.

### EXAMPLE 3

In a similar manner as in Example 1, a primary sequence NGNGNGNGNGLNYLKSLYGGYG (S3) was given and DNA sequences encoding this peptide sequences were generated. The total patterns of base sequence variants encoded in the first reading frame were approximately 87×10⁹. However, when the algorithm according to the “nucleic acid sequence-dipeptide corresponding table” of the present invention was applied, it was proved that the processing should only be carried out for about 57×10⁷ variants that had no translation termination codons in the second and third reading frames.

### INDUSTRIAL APPLICABILITY

The present invention makes it possible to design a multifunctional base sequence where the calculation time is largely shortened and the volume of memory consumption of a processor is largely reduced by calculating in a way that the base sequences are excluded in advance which are accompanied with the emergence of translation termination codons, which are to be excluded finally, in the second and third reading frames. The present invention also makes it possible to analyze translation products in the second and third reading frames without once back-translating peptide sequences to base sequences, and therefore, calculation speed of the algorithm which analyzes the property of peptides encoded by the same base sequence in different reading frames can largely be reduced and the memory consumption can be saved.

1. A method of designing a multifunctional base sequence wherein the base sequence has two or more functions in different reading frames of the base sequence, wherein a protein or a peptide encoded by a base sequence arising from one of the three reading frames is processed as a pool of oligopeptide units, and wherein the base sequence information of other reading frames contained in the oligopeptide sequence is utilized.

2. The method of designing a multifunctional base sequence according to claim 1, wherein a corresponding table for nucleic acid sequences encoding oligopeptide sequences is produced and used.

3. The method of designing a multifunctional base sequence according to claim 1 or 2, wherein a processing is carried out for a pool of sequential oligopeptide units having duplicated amino acid residues, and wherein a processing is carried out to connect oligopeptide units that have the same codon for the duplicated amino acid residue in the sequential oligopeptide units.

4. The method of designing a multifunctional base sequence according to claim 1 or 2, wherein a processing is carried out to connect amino acid residues encoded by base sequences of other reading frames contained in the oligopeptide units.

5. The method of designing a multifunctional base sequence according to any of claims 1-4, wherein the processing for a pool of oligopeptide units is a processing to exclude base sequences containing termination codons from among the base sequences of other reading frames contained in the oligopeptide units.

6. The method of designing a multifunctional base sequence according to any of claims 1-4, wherein the processing for a pool of oligopeptide units is a processing to select the whole or a part of a sequence of the interest from among the base sequences of other reading frames contained in the oligopeptide units.

7. The method of designing a multifunctional base sequence according to any of claims 1-6, wherein the base sequence is a double-stranded base sequence.

8. The method of designing a multifunctional base sequence according to any of claims 1-7, wherein the oligopeptide units are dipeptide units or tripeptide units.

9. A method of generating a multifunctional base sequence having two or more functions, wherein the method of designing a multifunctional base sequence according to any of claims 18 is employed.

10. A method of generating an artificial protein, wherein the method of designing a multifunctional base sequence according to any of claims 1-8 is employed.
